(19) United States
(12) Patent Application Publication
(10) Pub. No.: US 2020/0325454 A1
(43) Pub. Date: Oct. 15, 2020

(54) VECTORS CONTAINING AIMP2-DX2 AND TARGET NUCLEIC ACIDS FOR miR 142 AND USES THEREOF

(71) Applicant: GENEROATH CO., LTD, Seoul (KR)

(72) Inventor: Jin Woo CHOI, Seoul (KR)

(21) Appl. No.: 16/819,998

(22) Filed: Mar. 16, 2020

(30) Foreign Application Priority Data
Mar. 15, 2019 (KR) 10-2019-0030126

(51) Int. Cl. C12N 7/00; C07K 14/435; C12N 15/113; C12N 15/86; A61K 48/00

(52) U.S. Cl. CPC C12N 7/00; C07K 14/435; C12N 15/113; C12N 15/86; C12N 2750/14132; A61K 48/0066; C12N 2310/141; C12N 2750/14143; A61K 48/0075

(57) ABSTRACT

The invention relates to recombinant vectors comprising an AIMP2 splice variant and miR-142 target nucleic acid and its diverse range of applications. The AIMP2 variant can be used beneficially in relevant industries since it can specifically be expressed in neuronal cells and brain tissues.

### CROSS-REFERENCE TO RELATED APPLICATIONS

### Field of the Invention

This application claims priority to KR 10-2019-0030126, filed Mar. 15, 2019, which is incorporated by reference herein in its entirety.

### REFERENCE TO SEQUENCE LISTING SUBMITTED ELECTRONICALLY

The content of the electronically submitted sequence listing in ASCII text file (Name: 2493-0003US01—Sequence Listing_ST25.txt; Size: 6 KB; and Date of Creation: Mar. 16, 2020) filed with the application is incorporated herein by reference in its entirety.

### FIELD OF THE INVENTION

Disclosed herein are vector containing AIMP2-DX2 and a target sequence for miR-142 and uses thereof.

### BACKGROUND OF THE INVENTION

The brain of mammals can execute complex functions through establishment of systemic neural network after having undergone a series of processes including division, differentiation, survival and death of neuronal stem cells, and formation of synapses, etc. Neurons in the animal brain continuously produce a wide range of substances necessary in the growth of nerves even during their matured state, thereby inducing the growths of axon and dendrite. Moreover, it can be said that they continuously undergo differentiation since there is ceaseless synaptic remodeling of the neural network and synaptic connections whenever new learning and memorization is executed. Neurons undergo apoptosis if they are unable to receive target-derived survival factors such as neural growth factor in the process of cell differentiation and synaptic formation and apoptosis due to stress and cytotoxic agents become the main cause of degenerative cerebral disorders. When the peripheral nervous system of animals, unlike the central nervous system, is damaged, axons are regenerated over prolonged period of time. Axons at the back of the damaged nerves are degenerated by the process known as Wallerian degeneration and the cell body of the nerve recommences axonal regrowth while the Schwann cells are regenerated after having undergone a regeneration process, including determination of the target nerve through survival and extinction following division prior to undergoing differentiation, etc. again.

Throughout the world, there is a trend of continued increase in manifestation of neurodegenerative diseases every year along with the rapid increase in aged population. As the definitive prevention and treatment methods have not been discovered yet, there still is no drug with outstanding efficacy in treating such diseases. In addition, existing drugs and therapies used for these disorders frequently display side effects and toxicity arising from prolonged administration. Moreover, since they only have the effect of temporarily reducing the extent of symptoms or delaying the progress of the symptoms rather than complete treatment of the diseases, it is urgent to excavate and develop materials with decisive treatment efforts while reducing side effects and toxicity.

Approximately 600 cases of clinical trials on gene therapy on human subjects have been executed and were in progress until 2002 since the commencement of clinical trials in 1990 for the first time. On the foundation of the completion of human genome sequence analysis in 2003, development of new gene therapies will accelerate in the future through excavation of a diverse range of genes. However, 75% of the gene therapies that have been approved until now are targeted at monogenic diseases such as cancer or cystic fibrosis, etc., and there is no active development of gene therapy drugs for neural disorders or regeneration (Recombinant DNA consultation paper of NIH, USA (2002); Gene Therapy Clinical Trials, J. Gene Med. (2002) www.wiley.co.uk/genmed). Nonetheless, development of gene therapy by using neural growth factor such as NT-3 and glial derived neuronal factor (GDNF) for the treatment and regeneration of sensory neurons for Parkinson's disease is being attempted already (GDNF family ligands activate multiple events during axonal growth in mature sensory neurons (Mol. Cell. Neurosci. 25:4453-4459 (2004)). Since there is sluggish progress in the overall neuroscience researches on the cerebral functions in relation to disorders of nerve system, development of treatment drugs for various chronic disorders of nervous system is also confronted with difficulties.

AIMP2-DX2, as an alternative splicing variant of AIMP2, which is a tumor suppressor associate with apoptosis in many ways, is known to suppress apoptosis by hindering the functions of AIMP2. This is achieved by controlling TNF-a induced apoptosis by AIMP2/p38 mediated ubiquitination of TRAF2, and AIMP2-DX2, which is a splicing variant of AIMP2/p38 acting as competitive inhibitor of AIMP2 to promote the generation of tumor by suppressing TNF-a induced apoptosis through suppression of ubiquitination of TRAF2 and suppression of manifestation of Cox-2, which is an inflammation marker. In addition, it had been reported that AIMP2-DX2 has been confirmed as an existing lung cancer induction factor and, in the existing research, it was confirmed that AIMP2-DX2, which is a variant of AIMP2, manifested extensively in cancer cells induces cancer by interfering with the cancer suppression functions of AIMP2. Moreover, it was discovered that manifestation of AIMP2-DX2 in normal cell progresses cancerization of cells while suppression of manifestation of AIMP2-DX2, on the other hand, growth of cancer is suppressed, thereby displaying treatment effects.

It has also been determined that AIMP2-DX2 can be useful in treating neuronal diseases (KR10-2015-0140723 (2017) and US2019/0298858 (pub. Oct. 23, 2019).

### SUMMARY OF THE INVENTION

The recombinant vectors that include a sequence targeting miR-142, such as miR-142-3p and/or miR-142-5p target nucleic acids, can control the expression of the AIMP2 splicing variant, selectively in neuronal and brain tissues.

Disclosed herein are recombinant vectors comprising exon 2-deleted AIMP2 variant (AIMP2-DX2) gene and a miR-142 target nucleic acid.

The vectors can further comprise a promoter operably linked to the AIMP2-DX2. The promoter can be a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter or opsin promoter.

The miR-142 target nucleic acid can be 3′ to the AIMP2-DX2 gene. The miR-142 target nucleic acid can be 5′ to the AIMP2-DX2 gene.

The AIMP2-DX2 gene can have a nucleotide sequence encoding an amino acid sequence that is at least 90% identical to SEQ ID NO:2. The AIMP2-DX2 gene can have a nucleotide sequence encoding an amino acid sequence of SEQ ID NO:2.

The AIMP2-DX2 gene can have a nucleotide sequence at least 90% identical to a nucleotide sequence of SEQ ID NO:1. The AIMP2-DX2 gene can have a nucleotide sequence of SEQ ID NO:1.

The miR-142 target nucleic acid can comprise a nucleotide sequence comprising ACACTA. The miR-142 target nucleic acid can comprise a nucleotide sequence comprising ACACTA and 1-17 additional contiguous nucleotides of SEQ ID NO:5.

The miR-142 target nucleic acid can comprise a nucleotide sequence at least 50% identical to a nucleotide sequence of SEQ ID NO:5 (TCCATAAAGTAGGAAACACTACA; miR-142-3p). The miR-142 target nucleic acid can comprise a nucleotide sequence of SEQ ID NO:5.

The miR-142 target nucleic acid can comprise a nucleotide sequence comprising ACTTTA. The miR-142 target nucleic acid can comprise a nucleotide sequence comprising ACTTTA and 1-15 additional contiguous nucleotides of SEQ ID NO:7.

The miR-142 target nucleic acid can comprise a nucleotide sequence at least 50% identical to a nucleotide sequence of SEQ ID NO:7 (AGTAGTGCTTTCTACTTTATG; miR-142-5p). The miR-142 target nucleic acid can comprise a nucleotide sequence of SEQ ID NO:7.

The miR-142 target nucleic acid can be repeated 2-10 times.

The vector can be a viral vector. The viral vector can be an Adenovirus, Adeno-associated virus, Lentivirus, Retrovirus, Human immunodeficiency virus (HIV), MLU (Murine leukemia virus), ASLV (Avian sarcoma/leukosis), SNV (Spleen necrosis virus), RSV (Rous sarcoma virus), MMTV (Mouse mammary tumor virus), or Herpes simplex virus vector. The viral vector can be an adeno-associated virus (AAV), adeonovirus, lentivirus, retrovirus, vaccinia virus, or herpes simplex virus vector.

Also disclosed herein are methods of treating a neuronal disease in a subject in need thereof, comprising administering any of the vectors described herein.

The neuronal disease can be amyotrophic lateral sclerosis (ALS), Alzheimer's disease, Parkinson's disease, retinal degeneration, mild cognitive impairment, multi-infarct dementia, fronto-temporal dementia, dementia with Lewy bodies, Huntington's disease, degenerative neural disease, metabolic cerebral disorders, depression, epilepsy, multiple sclerosis, cortico-basal degeneration, multiple system atrophy, progressive supranuclear palsy, dentatorubropallidoluysian atrophy, spinocerebella ataxia, primary lateral sclerosis, spinal muscular atrophy, or stroke. The neuronal disease can be ALS.

The treatment can improve motor activity or prolongs lifespan of the subject.

The vectors can be administered to the brain or spinal cord. The vector can be administered to the brain by stereotaxic injection.

The purpose of the invention is to provide recombinant vector containing target sequence for miR-142-3p.

In addition, the invention can provide gene carrier including recombinant vector containing target sequence for miR-142-3p.

In addition, the invention can provide the method of delivering and expressing of heterogeneous gene in neuron that includes the stage of inputting the recombinant vector into entities.

In addition, the invention can provide 1) promoter; 2) base sequence that codes target protein linked with promoter to enable operation; and 3) expression cassette that includes the base sequence targeting miR-142-3p inserted into 3′UTR of the base sequence.

In addition, the invention can provide preventive or therapeutic preparation for neurodegenerative diseases that includes base sequence that codes AIMP-2 splicing variant with loss of exon 2 and base sequence that targets miR-142-3p linked to 3′UTR of the base sequence.

In order to accomplish the aforementioned purposes, the invention provides recombinant vector containing target sequence for miR-142-3p.

In addition, the invention provides gene carrier that includes recombinant vector containing target sequence for miR-142-3p.

In addition, the invention provides the method of delivering and expressing of heterogeneous gene in neuron that includes the stage of inputting the recombinant vector into entities.

In addition, the invention provides 1) promoter; 2) base sequence that codes target protein linked with promoter to enable operation; and 3) expression cassette that includes the base sequence targeting miR-142-3p inserted into 3′UTR of the base sequence.

In addition, the invention provides preventive or therapeutic preparation for neurodegenerative diseases that includes base sequence that codes AIMP-2 splicing variant with loss of exon 2 and base sequence that targets miR-142-3p linked to 3′UTR of the base sequence.

The recombinant vector of the invention has the effect of controlling the side effect of over-expression of AIMP2 splicing variant in the tumor by inserting miR-142-3p into the target sequence of the terminal end of AIMP2, and controlling the suppression of its expression in CD45-derived cells, in particular, lymphatic system and leukocytes. Therefore, it can be used beneficially in the relevant industries since the AIMP2 splicing variant can be expressed specifically only in neuron and selectively expressed only in the brain tissues among various tissues of the body.

### BRIEF DESCRIPTION OF THE FIGURES

FIG. 1 illustrates a recombinant vector of the invention.

FIG. 2 shows the nerve cell-specific expression effect of a recombinant vector of the invention under an in vitro environment.

FIG. 3 shows the nerve cell-specific expression effect of a recombinant vector of the invention under an in vivo environment.

FIG. 4 shows an miR142-3pT (target) sequence with 4 repeats of miR142-3pT (underlined).

FIG. 5A shows a schematic of miR142-3p with 1×, 2×, and 3× repeats, and mutant. FIG. 5B shows miR142-3p inhibition on DX2 expression with 1×, 2×, and 3× repeats of miR-142-3pT.

FIG. 6 shows that a core binding sequence is important in DX2 inhibition. A vector with Tseq×3 repeats, which showed significant inhibition of DX2 (FIG. 5B), and DX2 construct were used as controls. 100 pmol of miR-142-3p treatment inhibited Tseq×3 vector significantly but DX2 and mutant sequence were not inhibited.

FIG. 7 shows total RNA extracted from the spinal cord following intrathecal injection of scAAV2-DX2-miR142-3p. qRT-PCR was performed.

FIG. 8 shows nerve cell-specific expression effect of an expression vector of the invention under an in vitro environment.

### DETAILED DESCRIPTION OF THE INVENTION

AIMP2-DX2 is an alternative splice variant of the tumor suppressor AIMP2, which is associated with apoptosis. AIMP2-DX2 is known to inhibit apoptosis of tumors by suppressing the function of AIMP2.

KR 10-1067816 (2011) describes that an inhibitor of AIMP2-DX2 may treat inflammatory diseases. KR 10-1067816 (2011) also discloses that AIMP2/p38 promotes ubiquitination of TRAF2 to regulate TNF-alpha-induced apoptosis and that AIMP2-DX2, a splice variant of AIMP2/p38, serves as a competitive inhibitor of AIMP2 to inhibit the ubiquitin of TRAF2 and thus to inhibit TNF-alpha-induced apoptosis, thereby promoting tumor generation and inhibiting the expression of Cox-2, an inflammation marker.

In addition, AIMP2-DX2 has been previously identified as a lung cancer-inducing factor. In the study, it was found that AIMP2-DX2, which is a variant of AIMP2, is common in cancer cells and interferes with the cancer inhibitory function of AIMP2, thus causing cancer. It was also found that the expression of AIMP2-DX2 in normal cells leads to cell canceration whereas the inhibition of the development of AIMP2-DX2 inhibits the growth of cancer cells, resulting in cancer treatment effects. Also, the study showed through an animal model that inhibition of AIMP2-DX2 targets can lead to the treatment of ovarian cancer that does not respond to conventional anticancer drugs such as Taxol and cisplatin. However, AIMP2-DX2 itself does not have oncogenic ability to transform normal cells.

It has also been determined that AIMP2-DX2 can treat neuronal diseases (US2019/0298858 A1).

Disclosed herein are recombinant vectors comprising exon 2-deleted AIMP2 variant (AIMP2-DX2) gene and a miR-142 target nucleic acid. The vectors described herein can express DX2 specifically in neuronal cells but not in hematopoietic cells, such as leukocytes and lymphoid cells. Thus, the vectors described herein can be useful in specifically targeting neuronal cells for treating neuronal diseases.

The AIMP2-DX2 polypeptide (SEQ ID NO:2) is a splice variant of AIMP2 (SEQ ID NO: 3), in which the second exon (SEQ ID NO: 4) of AIMP2 is omitted. Specifically, the AIMP2-DX2 gene has a base sequence set forth in SEQ ID NO: 1, and the AIMP2-DX2 polypeptide has an amino acid sequence set forth in SEQ ID NO: 2.

In some embodiments, the AIMP2-DX2 gene can have a nucleotide sequence encoding an amino acid sequence that is at least 90% identical, at least 93% identical, at least 95% identical, at least 96% identical, at least 97% identical, at least 98% identical, at least 99% identical to SEQ ID NO:2, or any ranges of or % identity therein. The AIMP2-DX2 gene can have a nucleotide sequence encoding an amino acid sequence of SEQ ID NO:2.

The AIMP2-DX2 gene can have a nucleotide sequence at least 90% identical, at least 93% identical, at least 95% identical, at least 96% identical, at least 97% identical, at least 98% identical, at least 99% identical to a nucleotide sequence of SEQ ID NO:1, or any ranges of or % identity therein. The AIMP2-DX2 gene can have a nucleotide sequence of SEQ ID NO:1.

The miR-142 target nucleic acid can comprise a nucleotide sequence comprising ACACTA. The miR-142 target nucleic acid can comprise a nucleotide sequence comprising ACACTA and 1-17 additional contiguous nucleotides of SEQ ID NO:5. For example, the miR-142 target nucleic acid can comprise a nucleotide sequence comprising ACACTA and a sum of 1, 2, 3, 4, 5, 6, 7, 8, 9, 10, 11, 12, 13, 14, 15, 16, or 17 additional nucleotides that are contiguous 5′ or 3′ of ACACTA as shown in SEQ ID NO:5.

The miR-142 target nucleic acid can comprise a nucleotide sequence at least 50% identical, at least 60% identical, at least 70% identical, at least 80% identical, at least 90% identical, or at least 95% identical to a nucleotide sequence of SEQ ID NO:5 (TCCATAAAGTAGGAAACACTACA; miR-142-3p). The miR-142 target nucleic acid can comprise a nucleotide sequence of SEQ ID NO:5.

The miR-142 target nucleic acid can comprise a nucleotide sequence comprising ACTTTA. The miR-142 target nucleic acid can comprise a nucleotide sequence comprising ACTTTA and 1-15 additional contiguous nucleotides of SEQ ID NO:7. For example, the miR-142 target nucleic acid can comprise a nucleotide sequence comprising ACTTTA and a sum of 1, 2, 3, 4, 5, 6, 7, 8, 9, 10, 11, 12, 13, 14, or 15 additional nucleotides that are contiguous 5′ or 3′ of ACTTTA as shown in SEQ ID NO:7.

The miR-142 target nucleic acid can comprise a nucleotide sequence at least 50% identical, at least 60% identical, at least 70% identical, at least 80% identical, at least 90% identical, or at least 95% identical to a nucleotide sequence of SEQ ID NO:7 (AGTAGTGCTTTCTACTTTATG; miR-142-5p). The miR-142 target nucleic acid can comprise a nucleotide sequence of SEQ ID NO:7.

A microRNA (miRNA) is a non-coding RNA molecule that functions to control gene expression. miRNAs function via base-pairing with complementary sequences within mRNA molecules. miRNAs can bind to target messenger RNA (mRNA) transcripts of protein-coding genes and negatively control their translation or cause mRNA degradation. At present, more than 2000 human miRNAs have been identified and miRbase databases are publicly available. Many miRNAs are expressed in a tissue-specific manner and have an important roles in maintaining tissue-specific functions and differentiation.

Disclosed herein are recombinant vectors that can control the side effect of over-expression of the AIMP2-DX2 variant in a tumor by inserting miR-142-3p and/or miR-142-5p into the target sequence of a terminal end of AIMP2-DX2, and controlling suppression of AIMP2-DX2 expression in cd45-derived cells, in particular, the lymphatic system and leukocytes. Thus, the AIMP2-DX2 variant can be expressed only in neuronal cells or selectively expressed in brain tissues but not in other types of cells or tissues.

The invention provides recombinant vectors containing a target sequence for miR-142-3p and/or miR-142-5p. Disclosed herein are recombinant vectors comprising exon 2-deleted AIMP2 variant (AIMP2-DX2) gene and a miR-142-3p and/or miR-142-5p target nucleic acids.

The term “recombinant vector” refers to vector that can be expressed as the target protein or RNA in appropriate host cells, and gene construct that contains essential operably linked control factor to enable the inserted gene to be expressed appropriately.

The term “operably linked” refers to functional linkage between the nucleic acid expression control sequence and nucleic acid sequence that codes the targeted protein and RNA to execute general functions. For example, it can affect the expression of nucleic acid sequence that codes promoter and protein or RNA that has been linked for operability of the nucleic acid sequence. Operable linkage with recombinant vector can be manufactured by using gene recombinant technology, which is known well in the corresponding technology area, and uses generally known enzymes in the corresponding technology area for the area-specific DNA cutting and linkage.

The recombinant vectors can further comprise a promoter operably linked to the AIMP2-DX2. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, or opsin promoter.

The term “micro RNA (miRNA)” is a noncoding RNA composed of about 20, 21, 22, 23, or 24 nucleotides and plays the role of controlling gene expression. The miRNA acts at the post-transcription stage of the gene and, in the case of mammals, and it is known that approximately 60% of the gene expression is controlled by miRNA. miRNA plays an important role in a diverse range of processes within living body and has been disclosed to have correlation with cancer, cardiac disorders and nerve related disorders. miRNA, which is a single chain RNA, a target sequence of the miRNA can be used as long as the expression of the target gene among the 2-chain premature RNA can be suppressed. For example, miR-142-3p and miR-142-5p exist in miR-142 and any of the target nucleic acids thereof can be used in the invention. “miR-142” refers to both miR-142-3p and miR-142-5p and can desirably be miR-142-3p.

The miR-142 target nucleic acid can be 5′ or 3′ to the AIMP2-DX2 gene.

“miR-142-3p” can exist in the area at which translocation of its gene occurs in aggressive B cell leukemia and is known to express in hemopoietic tissues (bone marrow, spleen and thymus, etc.). In addition, miR-142-3p is known to be involved in the differentiation of hemopoietic system with confirmation of expression in the liver of fetal mouse (hemopoietic tissue of mouse).

In some embodiments, the miR-142-3p and/or miR-142-5p target nucleic acid is repeated at least 2-10 times, at least 2-8 times, at least 2-6 times, at least 4 times, or any range or number of times thereof.

As an example of the execution of the invention, the miR-142-3p can contain a base sequence indicated with number 3. In addition, sequence that targets miR-142-3p can contain a base sequence with sequence number of 4 that complimentarily binds with miR-142-3p. miR-142-3p target sequence of the invention that contains the complementary sequence is the base sequence indicated with number 5 but not limited to this.

The recombinant vector can additionally contain heterogeneous promoter and operably linked heterogeneous gene in the promoter.

“Heterogeneous gene” in the invention can include protein or polypeptide with biologically appropriate activation, and encrypted sequence of the targeted product such as immunogen or antigenic protein or polypeptide, or treatment activation protein or polypeptide.

Polypeptides can supplement deficiency or absent expression of endogenous protein in host cells. The gene sequence can be induced from a diverse range of suppliers including DNA, cDNA, synthesized DNA, RNA or its combinations. The gene sequence can include genome DNA that contains or does not contain natural intron. In addition, the genome DNA can be acquired along with promoter sequence or polyadenylated sequence. Genome DNA or cDNA can be acquired in various methods. genome DNA can be extracted and purified from appropriate cells through method publicly notified in the corresponding area. Or, mRNA can be used to produce cDNA by reverse transcription or other method by being separated from the cells. Or, polynucleotide sequence can contain sequence that is complementary to RNA sequence, for example, antisense RNA sequence, and the antisense RNA can be administered to individual to suppress expression of complementary polynucleotide in the cells of individuals.

Under the goals of the invention, the heterogeneous gene is an AIMP-2 splicing variant with loss of exon 2 and miR-142-3p target sequence of the invention can be linked to 3′ UTR of the heterogeneous gene. The sequence of the AIMP2 protein (312aa version: AAC50391.1 or GI: 1215669; 320aa version: AAH13630.1, GI: 15489023, BCO 13630.1) are described in literatures (312aa version: Nicolaides, N. C., Kinzler, K. W. and Vogelstein, B. Analysis of the 5′ region of PMS2 reveals heterogeneous transcripts and a novel overlapping gene, Genomics 29 (2), 329-334 (1995)/320 aa version: Generation and initial analysis of more than 15,000 full-length human and mouse cDNA sequences, Proc. Natl. Acad. Sci. U.S.A. 99 (26), 16899-16903 (2002)).

The term “AIMP2 splicing variant” of the invention refers to the variant generated due to partial or total loss of the exon 2 among the exons 1 to 4. As such, the variant signifies interference of the normal function of AIMP2 by forming AIMP2 protein and heterodimer. In addition, if the AIMP2 splicing variant is over-expressed in cells or tissues, cancer may be induced. Therefore, there is a need to induce tissue-specific expression in order to suppress induction of cancer.

The recombinant vector of the invention can include SEQ ID NOs: 1 and 5.

The term “% of sequence homology,” “% identity,” or “% identical” to a nucleotide or amino acid sequence can be, e.g., confirmed by comparing the 2 optimally arranged sequence with the comparison domain and some of the base sequences in the comparison domain can include addition or deletion (that is, gap) in comparison to the reference sequence on the optimal arrange of the 2 sequences (does not include addition or deletion).

Protein of the invention not only includes those with its natural type amino acid sequence but also those with variant amino acid sequence in the scope of the invention.

Variant of the protein of the invention signifies protein with difference sequence due to the deletion, insertion, non-conservative or conservative substitution or their combinations of natural amino acid sequence and more than 1 amino acid residue. Amino acid exchange in protein and peptide that does not modify the activation of the molecule in overall is notified in the corresponding area (H. Neurath, R. L. Hill, The Proteins, Academic Press, New York, 1979).

The protein or its variant can be manufactured through naturally extraction or synthesis (Merrifield, J. Amer. Chem. Soc. 85: 2149-2156, 1963) or gene recombination method on the basis of DNA sequence (Sambrook et al, Molecular Cloning, Cold Spring Harbour Laboratory Press, New York, USA, 2^{nd} Ed., 1989).

The amino acid mutation occurs on the basis of the relative similarity of the amino acid side chain substituent such as hydrophilicity, hydrophobicity, electric charge and size, etc. In accordance with the analysis of the size, shape and types of amino acid side chain substituent, it can be discerned that arginine, lysine and histidine are residues with positive charge; alanine, glycine and serine have similar sizes; phenylalanine, tryptophan and tyrosine have similar shapes. Therefore, on the basis of such considerations, arginine, lysine and histidine; alanine, glycine and serine; and phenylalanine, tryptophan and tyrosine can be deemed functional equivalents biologically.

In introducing one or more mutations, hydrophobic index of amino acid can be considered. Hydrophobic index is assigned to each amino acid according to hydrophobicity and charge: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5)

In assigning interactive biological function of protein, hydrophobic amino acid index is very important. It is a well-known fact that it is possible to have similar biological activation only if substitution is made with amino acid with similar hydrophobic index. In the event of introducing mutation by making reference to the hydrophobic index, execute substitution between amino acids with hydrophobic index differences within f 2 desirably, within f 1 more desirably and within 0.5 even more desirably.

Meanwhile, it is also well known that substitution between amino acids with similar hydrophilicity value induces proteins with equivalent biological activation. As indicated in the U.S. Pat. No. 4,554,101 in the USA, following hydrophilic values are assigned to each of the amino acid residues: arginine (+3.0); lysine (+3.0); aspartate (+3.0 f 1); glutamate (+3.0 f 1); serine (+0.3); asparagine (+0.2); glutamine (+0.2); glycine (0); threonine (−0.4); proline (−0.5 f 1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In the event of introducing one or more mutations by making reference to hydrophilic values, execute substitution between amino acids with hydrophilic value differences within f 2 desirably, within f 1 more desirably and within f 0.5 even more desirably.

Amino acid exchange in protein that does not modify the activation of molecule in overall is notified in the corresponding area (H. Neurath, R. L. Hill, The Proteins, Academic Press, New York, 1979). The most generally occurring exchanges are those between the amino acid residues including Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu and Asp/Gly. Vector system of the invention can be constructed through diverse methods announced in the corresponding industry. The specific methods are described by Sambrook et al. (2001), Molecular Cloning, A Laboratory Manual, Cold Spring Harbor Laboratory Press and this paper is specified as a reference in this statement of specifications.

Vectors of the invention can be constructed as a typical vector for cloning or for expression. In addition, vector of the invention can be constructed with prokaryotic or eukaryotic cells as the host. If the vector of the invention is an expression vector and prokaryotic cell is used as the host, it is general to include powerful promoter for execution of transcription (for example, tac promoter, lac promoter, lacUV5 promoter, 1pp promoter, pL X promoter, pRX promoter, rac5 promoter, amp promoter, recA promoter, SP6 promoter, trp promoter and T7 promoter, etc.), ribosome binding site for commencement of decoding and transcription/decoding termination sequence. In the case of using E. coli (for example, HB101, BL21, DH5a, etc.) as the host cell, promoter and operator site of the tryptophan biosynthesis route of E. coli (Yanofsky, C. (1984), J. Bacteriol., 158: 1018-1024) and left directional promoter of phage X (pLX promoter, Herskowitz, I. and Hagen, D. (1980), Ann. Rev. Genet., 14: 399-445) can be used as the control site.

Meanwhile, vectors that can be used in the invention can be more than 1 species selected from the group composed of virus vector, linear DNA and plasmid DNA.

“Virus vector” in the invention refers to the virus vector capable of delivering gene or genetic substance to the desired cells, tissue and/or organ.

Although the virus vectors can include more than 1 species from the group composed of Adenovirus, Adeno-associated virus, Lentivirus, Retrovirus, HIV (Human immunodeficiency virus), MLU (Murine leukemia virus), ASLV (Avian sarcoma/leukosis), SNV (Spleen necrosis virus), RSV (Rous sarcoma virus), MMTV (Mouse mammary tumor virus) and Herpes simplex virus, it is not limited to these. In some embodiments, the viral vector can be an adeno-associated virus (AAV), adeonovirus, lentivirus, retrovirus, vaccinia virus, or herpes simplex virus vector.

Although Retrovirus has the integration function for the genome of host cells and is harmless to human body, it has the characteristic including suppression of the functions of normal cells at the time of integration, and ability to infect a diverse range of cells, ease of proliferation, accommodate approximately 1-7 kb of external gene and generate duplication deficient virus. However, Retroviruses have the disadvantages including difficulties in infecting cells after mitotic division and gene delivery under an in vivo condition and need to proliferate somatic cells under in vitro condition. In addition, Retroviruses have the risk of sudden mutation as it can be integrated into proto-oncogene, thereby presenting the possibility of cell necrosis.

Meanwhile, Adenoviruses have various advantages as a cloning vector including duplication even in nucleus of cells in medium level size, clinically nontoxic, stable even if external gene is inserted, no rearrangement or loss of genes, transformation of eukaryotic organism and stably undergoes expression at high level even when integrated into host cell chromosome. Good host cells of Adenoviruses are the cells that are the causes of hemopoietic, lymphatic and myeloma in human. However, proliferation is difficult since it is a linear DNA and it is not easy to recover the infected virus along with low infection rate of virus. In addition, expression of the delivered gene is most extensive during 1-2 weeks with expression sustained over the 3-4 weeks only in some of the cells. Another issue is that it has high immuno-antigenicity.

Adeno-associated virus (AAV) has been preferred in recent years since it can supplement the aforementioned problems and has a lot of advantages as gene therapy agent. It is also referred as adenosatellite virus. Diameter of adeno-associated virus particle is 20 nm and is known to have almost no harm to human body. As such, its sales as gene therapy agent in Europe were approved.

AAV is a provirus with single strand that needs auxiliary virus for duplication and AAV genome has 4,680 bp that can be inserted into specific area of the chromosome 19 of the infected cells. Trans-gene is inserted into the plasma DNA connected by the 2 inverted terminal repeat (ITR) sequence section with 145 bp each and signal sequence section. Transfection is executed along with other plasmid DNA that expresses the AAV rep and cap sections, and Adenovirus is added as an auxiliary virus. AAV has the advantages of wide range of host cells that deliver genes, little immunological side effects at the time of repetitive administration and long gene expression period. Moreover, it is safe even if the AAV genome is integrated with the chromosome of host cells and does not modify or rearrange the gene expression of the host.

The Adeno-associated virus is known to have a total of 4 serotypes. Among the serotypes of many Adeno-associated viruses that can be used in the delivery of the target gene, the most widely researched vector is the Adeno-associated virus serotype 2 and is currently used in the delivery of clinical genes of cystic fibrosis, hemophilia and Canavan's disease. In addition, recently, the potential of recombinant adeno-associated virus (rAAV) is increasing in the area of cancer gene therapy [4]. It was also the Adeno-associated virus serotype 2 that was used in the invention. Although it is possible to select and apply appropriate viral vector, it is not limited to this.

In addition, if the vectors of the invention are expression vectors and use eukaryotic cells as the host, promoter derived from the genome of mammalian cells (example: metallothionein promoter) or promoter derived from mammalian virus (example: post-adenovirus promoter, vaccine virus 7.5K promoter, SV40 promoter, cytomegalovirus promoter and HSV TK promoter) can be used. Specifically, although it can include more than 1 species selected from the group composed of promoters selected from the group composed of LTR of Retrovirus, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter and opsin promoter, it is not limited to these. Moreover, it generally has polyadenylated sequence as the transcription termination sequence.

Vectors of the invention can be fused with other sequences as need to make the purification of the protein easier. Although the fused sequence such as glutathione S-transferase (Pharmacia, USA), maltose binding protein (NEB, USA), FLAG (MI, USA) and 6×His (hexahistidine; Quiagen, USA), etc. can be used, for example, it is not limited to these. In addition, expression vector of the invention can include tolerance gene against antibiotics generally used in the corresponding industry as the selective marker including Ampicillin, Gentamycin, Carbenicillin, Chloramphenicol, Streptomycin, Kanamycin, Geneticin, Neomycin and Tetracycline, as examples.

In addition, the invention provides gene carriers including the recombinant vector containing target sequence for miR-142, such as miR-142-3p and/or miR-142-5p.

The term “gene transfer” in the invention includes delivery of genetic substances to cells for transcription and expression in general. Its method is ideal for protein expression and treatment purposes. A diverse range of delivery methods such as DNA transfection and virus transduction are announced. It signifies virus-mediated gene transfer due to the possibility of targeting specific receptor and/or cell types through high delivery efficiency and high level of expression of delivered genes, and, if necessary, nature-friendliness or pseudo-typing.

The gene carriers can be transformed entity that has been transformed into the recombinant vector of the invention, and transformation includes all methods of introducing nucleic acid to organic entity, cells, tissues or organs, and, as announced in the corresponding area, it is possible to select and execute appropriate standard technology in accordance with the host cells. Although such methods include electroporation, fusion of protoplasm, calcium phosphate (CaPO₄) sedimentation, calcium chloride (CaCl₂) sedimentation, mixing with the use of silicone carbide fiber, agribacteria-mediated transformation, PEG, dextran sulphate and lipofectamin, etc., it is not limited to these.

The gene carriers are for the purpose of expression of heterogeneous genes in neuron. As such it suppresses the expression of the heterogeneous gene in CD45-derived cells and can increase the expression of heterogeneous gene in brain tissue. Majority of the CD45 are transmembrane protein tyrosine phosphatase situated at the hematopoietic cell. Cells can be defined in accordance with the molecules situated on the cell surface and the CD45 is the cell marker for all leukocyte groups and B lymphocytes. The gene carrier of the invention may not be expressed in the CD45-derived cells, in particular, in lymphoid and leukocyte range of cells.

The gene carriers can additionally include carrier, excipient or diluent allowed to be used pharmacologically.

In addition, the invention provides methods of delivering and expressing the heterogeneous gene in the neuron that includes the stage of introducing the recombinant vector into the corresponding entity.

The methods can increase the expression of heterogeneous gene in cerebral tissues and control heterogeneous gene expression in other tissues.

In addition, the invention provides 1) promoter; 2) base sequence that codes target protein linked with promoter to enable operation; and 3) expression cassette that includes the base sequence targeting miR-142-3p inserted into 3′UTR of the base sequence. The invention provides 1) promoter; 2) base sequence that codes target protein linked with promoter to enable operation; and 3) expression cassette that includes the base sequence targeting miR-142-5p inserted into 3′UTR of the base sequence.

The term “expression cassette” in the invention refers to the unit cassette that can execute expression for the production and secretion of the target protein operably linked with the downstream of signal peptide as it includes gene that codes the target protein and base sequence that odes the promoter and signal peptide. Secretion expression cassette of the invention can be used mixed with the secretion system. A diverse range of factors that can assist the efficient production of the target protein can be included in and out of such expression cassette.

In addition, the invention provides preventive or therapeutic preparation for neurodegenerative diseases that includes base sequence that codes AIMP-2 splicing variant with loss of exon 2 and base sequence that targets miR-142-3p linked to 3′UTR of the base sequence.

Accordingly, also disclosed herein are methods of treating a neuronal disease in a subject in need thereof, comprising administering any of the vectors disclosed herein. Although the neurodegenerative diseases can be more than 1 of the diseases selected from the group composed of Alzheimer's disease, Parkinson's disease, amyotrophic lateral sclerosis (ALS), retinal degeneration, mild cognitive impairment, multi-infarct dementia, fronto-temporal dementia, dementia with Lewy bodies, Huntington's disease, degenerative neural disease, metabolic cerebral disorders, depression, epilepsy, multiple sclerosis, cortico-basal degeneration, multiple system atrophy, progressive supranuclear palsy, dentatorubropallidoluysian atrophy, spinocerebella ataxia, primary lateral sclerosis, spinal muscular atrophy and stroke, it is not limited to these. In some embodiments, the neuronal disease is ALS. The treatment can improve memory, dyskinesia, motor activity, and/or prolong lifespan of the subject with a neuronal disease, e.g., ALS, Alzheimer's disease, or Parkinson's disease. In some embodiments, the treatment can improve motor activity and/or prolong lifespan of the subject with a neuronal disease, e.g., ALS.

The vectors disclosed herein can effect, but not limited to, apoptosis inhibition, dyskinesia amelioration, and/or oxidative stress inhibition, and thus prevent or treat neuronal diseases.

The term “treatment” in the invention includes not only complete treatment of neurodegenerative diseases but also partial treatment, improvement and reduction in the overall symptoms of neurodegenerative diseases as the results of application of the pharmacological agent in accordance with the invention to the entity with degenerative cerebral disorders.

The term “prevention” in the invention signifies prevention of the occurrence of overall symptoms of neurodegenerative diseases in advance by suppressing or blocking the symptoms or phenomenon such as cognition disorder, behavior disorder and destruction of brain nerves by applying pharmacological agent in accordance with the invention to the entity with degenerative cerebral disorders.

Adjuvants other than the active ingredients can be included additionally to the pharmacological agent in accordance with the invention. Although any adjuvant can be used without restrictions as long as it is known in the corresponding technical area, it is possible to increase immunity by further including complete and incomplete adjuvant of Freund, for example.

Pharmacological agents in accordance with the invention can be manufactured in the format of having mixed the active ingredients with the pharmacologically allowed carrier. Here, pharmacologically allowed carrier includes carrier, excipient and diluent generally used in the area of pharmacology. Pharmacologically allowed carrier that can be used for the pharmacological agent in accordance with the invention include lactose, dextrose, sucrose, sorbitol, mannitol, xylitol, erythritol, malitol, starch, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil, but not limited to these.

Pharmacological agents in accordance with the invention can be used by being manufactured in various formats including oral administration types such as powder, granule, pill, capsule, suspended solution, emulsion, syrup and aerosol, etc., and external application, suppository drug or disinfection injection solution, etc. in accordance with their respective general manufacturing methods.

When manufactured into preparations, diluents or excipients such as filler, extender, binding agent, humectant, disintegrating agent and surfactant, etc., which are used generally, can be used in the manufacturing. Solid preparations for oral administration include pill, tablet, powder, granule and capsule preparations, and such solid preparations can be manufactured by mixing more than 1 excipient such as starch, calcium carbonate, sucrose, lactose and gelatin with the active ingredients. In addition, lubricants such as magnesium stearate and talc can also be used in addition to simple excipients. Liquid preparations for oral administration include suspended solution, solution for internal use, oil and syrup, etc. with the inclusion of various excipients such as humectant, sweetening agent, flavoring and preservative, etc. other than water and liquid paraffin, which are the generally used diluents. Preparations for non-oral administration include sterilized aqueous solution, non-aqueous solvent, suspension agent, oil, freeze dried agent and suppository. Vegetable oil such as propylene glycol, polyethylene glycol and olive oil, and injectable esters such as ethylate can be used as non-aqueous solvent and suspension solution. Agents for suppository can include witepsol, tween 61, cacao oil, laurine oil and glycerogelatin, etc.

Pharmacological agents in accordance with the invention can be administered into entity through diversified channels. All formats of administration such as oral administration, and intravenous, muscle, subcutaneous and intraperitoneal injection can be anticipated.

Desirable doses of administration of therapeutic agents in accordance with the invention differs depending on various factors including preparation production method, administration format, age, weight and gender of the patient, extent of the symptoms of the disease, food, administration period, administration route, discharge speed and reaction sensitivity, etc. Nonetheless, it can be selected appropriately by the corresponding manufacturer.

However, for the treatment effects, skilled medical doctor can determine and prescribe effective dose for the targeted treatment. For example, the treatment agents include intravenous, subcutaneous and muscle injection, and direction injection into cerebral ventricle or spinal cord by using micro-needle. Multiple injections and repetitive administrations are possible, e.g., the effective dose is 0.05 to 15 mg/kg in the case of vector, 5×10¹¹ to 3.3×10¹⁴ viral particle (2.5×10¹² to 1.5×10¹⁶ IU)/kg in the case of recombinant virus and 5×10² to 5×10⁷ cells/kg in the cells. Desirably, the doses are 0.1 to 10 mg/kg in the case of vector, 5×10¹² to 3.3×10¹³ particles (2.5×10¹³ to 1.5×10¹⁵ IU)/kg in the case of recombinant virus and 5×10³ to 5×10⁶ cells/kg in the case of cells at the rate of 2 to 3 administrations per week. The dose is not strictly restricted. Rather, it can be modified in accordance with the condition of the patient and the extent of manifestation of the neural disorders. Effective dose for other subcutaneous fat and muscle injection, and direct administration into the affected area is 9×10¹⁰ to 3.3×10¹⁴ recombinant viral particles with the interval of 10 cm and at the rate of 2-3 times per week. The dose is not strictly restricted. Rather, it can be modified in accordance with the condition of the patient and the extent of manifestation of the neural disorders. More specifically, pharmacological agent in accordance with the invention includes 1×10¹⁰ to 1×10¹² vg (virus genome)/mL of recombinant adeno-associated virus and, generally, it is advisable to inject 1×10¹² vg once every 2 days over 2 weeks. It can be administered once a day or by dividing the dose for several administrations throughout the day.

The pharmacological preparations can be produced in a diverse range of orally and non-orally administrable formats. In some embodiments, the vector disclosed herein can be administered to the brain or spinal cord. In some embodiments, the vectors disclosed herein can be administered to the brain by stereotaxic injection.

Orally administrative agents include pills, tablets, hard and soft capsules, liquid, suspended solution, oils, syrup and granules, etc. These agents can include diluent (example: lactose, dextrose, sucrose, mannitol, sorbitol, cellulose and/or glycine) and glydents (example: silica, talc, and stearic acid and its magnesium or calcium salts, and/or polyethylene glycol) in addition to the active ingredients. In addition, the pills can contain binding agents such as magnesium aluminum silicate, starch paste, gelatin, tragacanthin, methyl cellulose, sodium carboxymethyl cellulose and/or polyvinyl pyrrolidine, and, depending on the situation, can contain disintegration agent such as starch, agar, alginic acid or its sodium salt or similar mixture and/or absorbent, coloring, flavor and sweetener. The agents can be manufactured by general mixing, granulation or coating methods.

In addition, injection agents are the representative form of non-orally administered preparations. Solvents for such injection agents include water, Ringer's solution, isotonic physiological saline and suspension. Sterilized fixation oil of the injection agent can be used as solvent or suspension medium, and any non-irritating fixation oil including mono- and di-glyceride can be used for such purpose. In addition, the injection agent can use fatty acids such as oleic acid.

Additional embodiments A-X follow.

A. Recombinant vector containing miR-142-3p targeted sequence.

B. With regards to the embodiment A, recombinant vector for which the miR-142-3p is indicated with base sequence number of 3.

C. With regards to the embodiment A, recombinant vector for which the miR-142-3p targeted sequence is the sequence that binds with miR-142-3p sequence complementarily.

D. With regards to the embodiment A, recombinant vector for which the miR-142-3p targeted sequence is indicated with base sequence number of 4.

E. With regards to the embodiment A, recombinant vector for which the miR-142-3p targeted sequence is indicated with base sequence number of 5.

F. With regards to the embodiment A, recombinant vector for which the miR-142-3p targeted is indicated with base sequence number of 4 and base sequence with more than 90% homology.

G. With regards to the embodiment A, recombinant vector for which the miR-142-3p targeted is indicated with base sequence number of 5 and base sequence with more than 90% homology.

H. With regards to the embodiment A, recombinant vector for which the recombinant vector additionally includes heterogeneous gene operably linked to the heterogeneous promoter and promoter.

I. With regards to the embodiment H, recombinant vector for which miR-142-3p targeted sequence has been inserted into the 3′ UTR of the heterogeneous gene.

J. With regards to the embodiment H, recombinant vector for which the heterogeneous gene is an AIMP2 variant with deletion of exon 2.

K. With regards to the embodiment H, recombinant vector for which the heterogeneous promoter has been selected from the group consisting of Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter and opsin promoter.

L. With regards to the embodiment A, recombinant vector for which the recombinant vector is more than 1 type selected from the group consisting of virus vector, linear DNA and plasmid DNA.

M. With regards to the embodiment L, recombinant vector for which the virus vector is a vector derived from more than 1 species selected from the group consisting of Adenovirus, Adeno-associated virus, Lentivirus, Retrovirus, Human immunodeficiency virus (HIV), MLU (Murine leukemia virus), ASLV (Avian sarcoma/leukosis), SNV (Spleen necrosis virus), RSV (Rous sarcoma virus), MMTV (Mouse mammary tumor virus) and Herpes simplex virus.

N. With regards to the embodiment A, recombinant vector for which the recombinant vector includes base sequence indicated with sequence number of 7.

O. Gene carrier that includes recombinant vector containing target sequence for miR-142-3p.

P. With regards to the embodiment O, gene carrier for which the gene carrier expresses heterogeneous gene in neuron.

Q. With regards to the embodiment O, gene carrier for which the gene carrier suppresses the expression of heterogeneous gene in CD45-derived cells.

R. With regards to the embodiment O, gene carrier for which the gene carrier increases the expression of heterogeneous gene in brain tissue.

S. Method of delivering and expressing heterogeneous gene to and in neuron including the stage of administering the recombinant vector of the embodiment A above into the entity.

T. With regards to the embodiment S, method of increasing the expression of heterogeneous gene and controlling the expression of heterogeneous genes in other tissues.

U. Embodiment comprising:
1) Promoter;
2) Base sequence that codes the target protein operably lined to promoter; and
3) Expression cassette that includes miR-142-3p target base sequence inserted into 3′UTR of the base sequence.

V. With regards to the embodiment U, expression cassette for which the target protein is an AIMP-2 variant protein with deleted exon 2.

W. Preventive or treatment preparations for neurodegenerative diseases with base sequence that codes AIMP-2 variant with deleted exon 2 and includes the miR-142-3p target base sequence inserted into 3′UTR of the base sequence.

X. With regards to the embodiment W, the neurodegenerative diseases is more than one of the diseases selected from the group consisting of Alzheimer's disease, Parkinson's disease, Lou Gehrig's disease (amyotrophic lateral sclerosis), retinal degeneration, mild cognitive impairment, multi-infarct dementia, fronto-temporal dementia, dementia with Lewy bodies, Huntington's disease, degenerative neural disease, metabolic cerebral disorders, depression, epilepsy, multiple sclerosis, cortico-basal degeneration, multiple system atrophy, progressive supranuclear palsy, dentatorubropallidoluysian atrophy, spinocerebella ataxia, primary lateral sclerosis, spinal muscular atrophy and stroke, it is not limited to these.

The invention will be explained in more detail by using the following execution examples below. However, the following execution examples are only for the purpose of specifying the contents of the invention and do not limit the application of the invention to such examples.

### EXAMPLES

### Example 1. Production of the Recombinant Vector

Majority of CD45 are transmembrane protein tyrosine phosphatase of the hematopoietic cell, which can be used to define the cells in accordance with the molecule on the cell surface. The CD45 is the marker for all the leukocyte groups and B lymphocytes. The inventors produced recombinant vector that is expressed specifically and only in neuron without being expressed in CD45-derived cells, in particular, lymphoid and leukocyte cell ranges. The recombinant vector contains a splicing variant for which the exon 2 of the Aminoacyl tRNA Synthetase Complex Interacting Multifunctional Protein 2 (AIMP2) has been deleted and by inserting miRNA capable of controlling the expression of the AIMP2 splicing variant.

It was confirmed that the AIMP2 splicing variant is over-expressed in the tumor by interfering with action of suppression of tumor of AIMP2 as AIMP2 splicing variant, which does not have the function to degrade TRAF2 (TNF receptor-associated factor 2) related with signal transduction of tumor by having executed splicing of AIMP2 hinders the functions of AIMP2 by competing with AIMP2 in binding with TRAF2.

Therefore, the recombinant vector of the invention was produced as above in order to induce specific expression of the AIMP2 splicing variant only in neuron and suppress expression of the AIMP2 splicing variant in the tumor.

1-1. Production of AIMP2 Variant

AIMP2 is one of the proteins involved in the formation of aminoacyl-tRNA synthetase (ARSs) and acts as a tumor suppressor. In order to construct plasmid that express the variant for which the exon 2 of the AIMP2 has been deleted, cDNA of AIMP2 splicing variant was cloned with pcDNA3.1-myc. The sub-cloning in pcDNA3.1-myc was executed by using EcoR1 and Xho 1 after having amplified AIMP2 splicing variant by using primer with attached EcoR 1 and Xho 1 linker to H322 cDNA.

AIMP2 variant of the invention has a nucleotide sequence of SEQ ID NO:1 and an amino acid sequence of SEQ ID NO:2.

1-2. Sorting of miRNA and Selection of its Target Sequence

As mentioned above, it was confirmed that the AIMP2 variant of the invention is over-expressed in tumor. As such, miRNA and its target capable of controlling the AIMP2 variant expression was selected in order to suppress expression of AIMP2 variant in leukocyte and lymphoid related cells while at the same time expressed safely in the neuron, the target cell.

For this purpose, miR-142-3p that is specifically expressed only in hematopoietic cells that generate leukocyte and lymphoid related cells was selected as the target. In order to produce the sequence that targets only the miR-142-3p, microarray data of mouse B cells and computer programming of genes targeted by miR⁻142-3p (mirSVR score) were used. The miR-142-3p is a base sequence indicated with the sequence number of 3. The sequence targeting miR-142-3p was indicated with base sequence number of 4 that binds with miR-142-3p complementarily. MiR-142-3p target sequence can have a nucleotide sequence of SEQ ID NO:5.

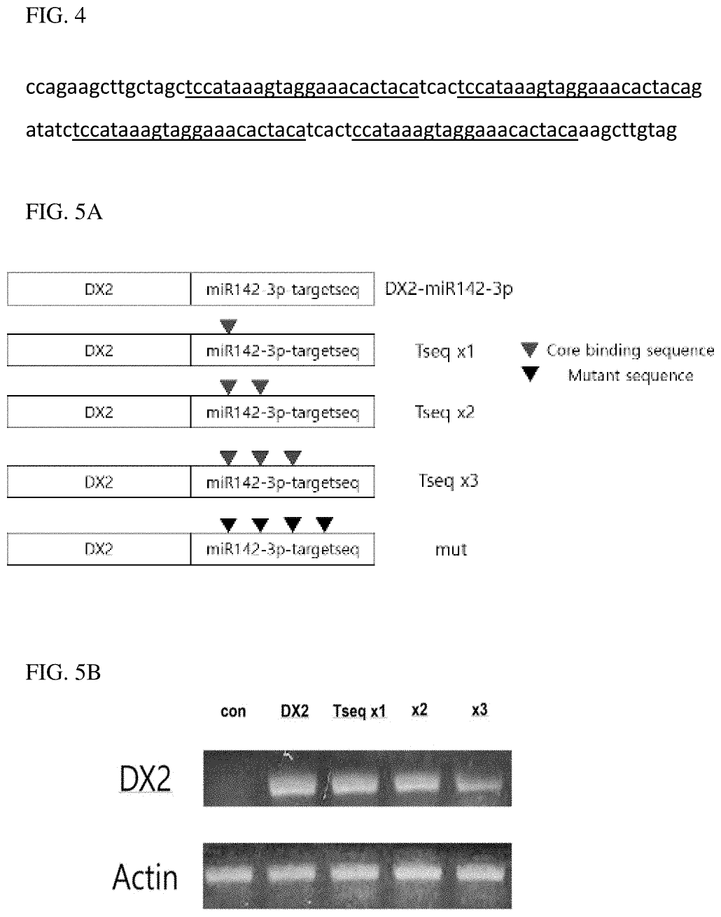

The miR-142-3p target sequence of the invention includes limiting enzyme for cloning (Nhe 1 and Hind III, Bmt 1) site sequence (ccagaagcttgctagc) and limiting enzyme (Hind H) site sequence (aagcttgtag). It includes the nucleotide sequence of SEQ ID NO:5 that has been repeated 4 times with the linkers (tcac and gatatc) that connects them (FIG. 4; SEQ ID NO:6).

1-3. Production of the Recombinant Vector

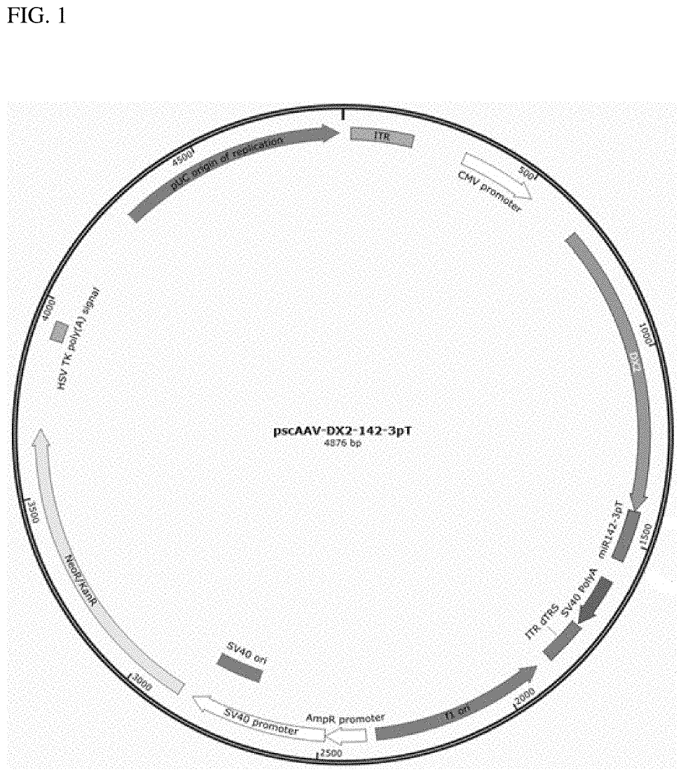

In order to produce the recombinant vector of the invention, miR-142-3p target sequence (SEQ ID NO:5) was inserted into 3′UTR of the AIMP2 variant (sequence number of 1) of the invention. Connecting of the AIMP-2 variant and miR-142-3p target sequence is indicated with base sequence number of 6, and, specifically, was cut and inserted by using Nhe I and Hind III sites. The recombinant vector is shown in FIG. 1.

### Example 2. Confirmation of the Nerve Cells Specific Expression of Recombinant Vector

2-1. Confirmation of Neuron-Specific Expression Effect Under In Vitro Condition

Since miR142-3p is specifically expressed only in hemopoietic cells, the extent of the expression of AIMP2 variant was confirmed in specific cells in accordance with the knockdown of AIMP2 variant of the invention according to the expression of miR142-3p target sequence of the recombinant vector of the invention.

Specifically, there were group with no treatment of the recombinant vector of the invention (SHAM), void/control vector processed group (NC vector), single AIMP2 variant vector processed group (pscAAV_DX2) and group treated with the recombinant vector of the invention (pscAAV-DX2-miR142-3pT). The concentration of all the vectors is in the unit of ug/ul and each group was treated with 2.5 ul (2.5 ug). In each of the treatment groups, treatments were made on the THP-1 cells strain (human leukemic monocyte cells) and SH-SY5Y cells strain (neuroblastoma) with confirmation of knockdown of AIMP2 variant. qPCR was executed by using the primers in the Table 1 below (degeneration for 15 seconds, and annealing and extension over 40 cycles under the temperature of 60° C. for 30 seconds).

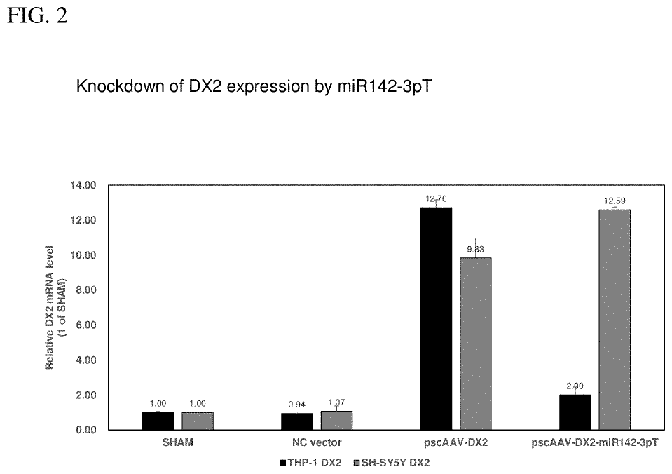

As the result, it was confirmed that AIMP2 variant is not expressed in the SHAM and NC vector groups. In addition, it was confirmed that there was expression in both the THP-1 cell strain and SH-SY5Y cell strain of the single AIMP2 variant vector processed group (pscAAV-DX2), thereby confirming that nerve cell-specific expression is not induced. On the other hand, it was confirmed that the AIMP2 variant is specifically expressed only in the SH-SY5Y cell strain in the group treated with the recombinant vector of the invention (FIG. 2).

TABLE 1

| AIMP2 | | SEQ |
|---|---|---|
| variant | Primer | ID NO: |
| Forward | CTGGCCACGTGCAGGATTACGGGG | 8 |
| | (only human) | |
| Reverse | AAGTGAATCCCAGCTGATAG | 9 |
| | (only human) | |

2-2. Confirmation of Nerve Cell-Specific Expression Effect Under In Vivo Conditions

Specifically, there were void/control vector processed group (NC vector), single AIMP2 variant vector treated group (pscAAV-DX2) and group treated with the recombinant vector of the invention (pscAAV-DX2-miR142-3pT). Intraparenchymal treatment with 10 ul (10⁹ vg) each of the virus with concentration of 10⁸ vg/ul was executed. After the intracranial injection of each of the treated groups into the mouse, expression of AIMP2 was confirmed in large intestinal tissues, lung tissues, cerebral tissues, hepatic tissues, renal tissues, thymus tissues, spleen tissues and peripheral blood mononuclear cells (PBMC) after 1 week. qPCR was executed by using the primers in the Table 1 below (degeneration for 15 seconds, and annealing and extension over 40 cycles under the temperature of 60° C. for 30 seconds).

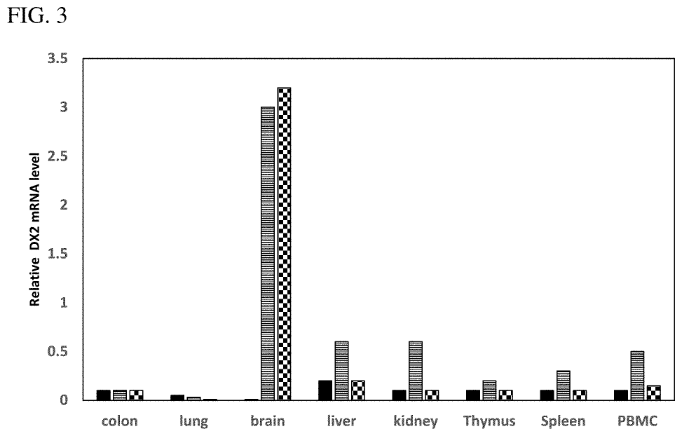

As the results, it was confirmed that the expression of AIMP2 variant specifically increased only in the brain tissue with highly concentrated neurons in the group treated with the recombinant vector of the invention (FIG. 3). On the other hand, it was confirmed that the expression of AIMP2 variant is hindered in tissues other than the brain tissue.

### Example 3. Materials and Methods

3-1. qRT-PCR

Total RNA was isolated from spinal cord using TRIzol (Invitrogen, Waltham, Mass., USA) according to the manufacturer's protocol. The extracted RNA was quantified by a spectrophotometer (ASP-2680, ACTgene, USA) for quantification. For making cDNA, a reverse transcription was performed using the SuperScript III First-Strand (Invitrogen) through manufacturer's protocol. The resulting cDNA was used for real-time PCR using SYBR green PCR master mix (ThermoFisher Scientific, USA). Expression data of the duplicated result were used for 2-AACt statistical analysis and GADPH expression was used for normalization.

3-2. Animals

hSOD1 G93A transgenic mice (B6.Cg-Tg(SOD1*G93A)1Gur/J) used in this study were purchased from the Jackson Laboratories (Bar Harbor, Me., USA). Age matched WT control mice were also used. The animals were housed in individual cages under specific pathogen-free conditions and a constant environment condition (21-23° C. temperature, 50-60% humidity and 12-h light/dark cycle) in the animal facility of Seoul National University, Republic of Korea. All experimental procedures were performed in accordance with guidelines of the Seoul National University Institutional Animal Care and Use Committee (SNUIACUC, Aug. 7, 2017) and this study was approved by our local ethic committee “SNUIACUC” (Approval No. SNU-170807-1). In pre-symptomatic stage, same age, female mice were administrated with AAV-GFP and DX2 vector. AAV-DX2 transduction were intrathecally injected by direct lumber puncture. Total 80 (4 μl/point) of AAV-GFP or DX2 vector with a Hamilton syringe (Hamilton, Switzerland) was slowly injected (1 μl/min) at two points while the needle was slowly retracted to prevent loss of injected vector.

3-3. miR142-3p Inhibition Experiment

miR-142-3p inhibition on DX2 expression could be observed from ×1 miR-142-3p target sequence. The HEK293 cells were transiently transfected with the ×1, ×2, and ×3 repeat miR-142-3p target sequence vectors, and also with 100 pmol miR-142-3p using lipofectamine 2000 (Invitrogen, US), and then incubated for 48 hrs. The amount of DX2 mRNA was analyzed by PCR. miR142-3p inhibition on DX2 expression was observed from Tseq×1 repeat miR142-3p target seq (FIG. 5B).

### Example 4

4-1. 3 Types of Vectors Generated for Inhibition Effect of Core Binding Sequence

Tseq×1 contains 1 core binding sequence, Tseq×2 contains 2 core binding sequences, and Tseq×3 contains 3 core binding sequences (FIG. 5A).

miR142-3p (100 pmol) inhibition on DX2 expression was started to be observed from ×1 repeat miR142-3p target sequence. The HEK293 cells were transiently transfected with the ×1, ×2, and ×3 repeat miR-142-3p T seq vectors, and also with 100 pmol miR-142-3p using lipofectamin 2000 (invitrogen, US), then incubated for 48 h. Amount of DX2 mRNA was analyzed by PCR. When the number of core binding sequence in miR142-3p target seq are increased, miR142-3p inhibition on DX2 expression was also increased. Tseq×3 core sequence containing vector showed significant inhibition (FIG. 5B).

4-2. Core Sequence Mutation.

Using mouse B cell microarray data and mirSVR score of miR-142-3p target gene, core sequence was predicted. Four regions of core sequences were substituted as follows: (5′-AACACTAC-3′ [symbol] 5′-CCACTGCA-3′) (see FIG. 4 for original sequence and FIG. 5A for schematic drawing).

4-3. Core Binding Sequence is Important DX2 Inhibition

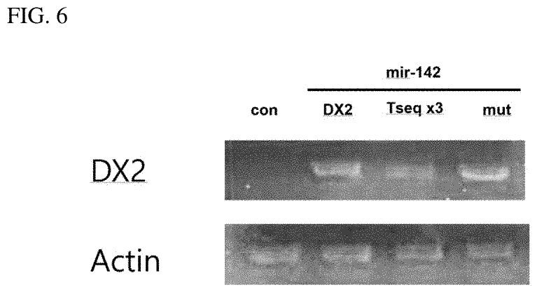

Four core sequences were substituted (FIG. 5A). The HEK293 cells were transiently transfected with the DX2-miR-142-3p T seq×3 repeated vector (Tseq3×) or with core sequence mutated vector (mut), and with 100 pmol miR-142-3p by using lipofectamin 2000 (Invitrogen, US), and then incubated for 48 hrs. Expression of DX2 mRNA was analyzed by PCR. Tseq×3 repeated vector which showed significant inhibition of DX2 (FIG. 5B) and DX2 construct were used as control. 100 pmol of miR142-3p treatment inhibited Tseq×3 vector significantly but DX2 and mut sequence were not inhibited (FIG. 6).

4-4. Tissue Distribution Data in ALS Mouse Model.

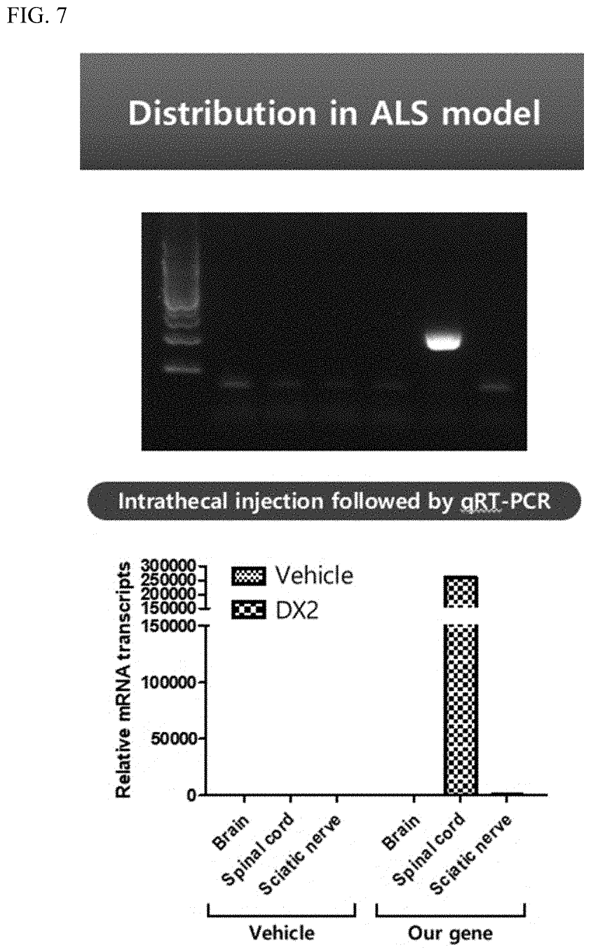

Total RNA from the spinal cord was extracted following intrathecal injection of the scAAV2-DX2-miR142-3p. qRT-PCR was performed. DX2 expression should be limited only in the local injection site, the spinal cord. hSOD1 G93A transgenic mice, scAAV-DX2 miR142-3p was expressed with intrathecal injection. Control vehicle injection showed expression only in spinal cord, not brain nor sciatic nerve (FIG. 7).

### Example 5

In Example 2, HEK293T cells were co-transfected with the three plasmids from Oxgene, UK, that encode all the components necessary to produce recombinant AAV2 particles.

HEK293T cells were also transfected with only pSF-AAV-ITR-CMV-EGFP-ITR-KanR (Oxgene, UK) with an insertion of AIMP2-DX2 or DX2-miR142 target nucleotide as expression vectors and not for producing AAV particles.

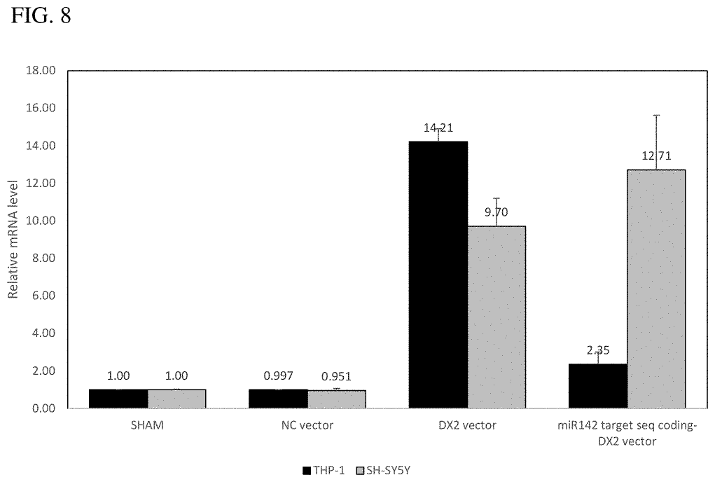

DX2 coding vector (2 ug) and DX2-miR142 target seq coding vector (2 ug) were transfected into THP-1 cell (human monocyte, CD45+ cell) and SH-SY5Y (neuronal cell). After 48 hrs, the cells were harvested and mRNA was isolated. With the synthesized cDNA, the expression of DX2 was analyzed by real-time PCR.

Whereas DX2 expression level was similar between DX2 coding vector and DX2-miR142 target seq coding vector transfected SH-SY5Y, DX2 expression was dramatically decreased in DX2-miR142 target seq coding vector transfected THP-1. Thus, miR142-3p worked only in THP-1 cells (FIG. 8).

The foregoing description of the specific embodiments will so fully reveal the general nature of the invention that others can, by applying knowledge within the skill of the art, readily modify and/or adapt for various applications, without departing from the general concept of the invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

1. A recombinant vector comprising exon 2-deleted AIMP2 variant (AIMP2-DX2) gene and a miR-142 target nucleic acid.

2. The vector of claim 1, further comprising a promoter operably linked to the AIMP2-DX2.

3. The vector of claim 2, wherein the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter or opsin promoter.

4. The vector of claim 1, wherein the miR-142 target nucleic acid is 3′ to the AIMP2-DX2 gene.

5. The vector of claim 1, wherein the AIMP2-DX2 gene has a nucleotide sequence encoding an amino acid sequence that is at least 90% identical to SEQ ID NO:2.

6. The vector of claim 5, wherein the AIMP2-DX2 gene has a nucleotide sequence encoding an amino acid sequence of SEQ ID NO:2.

7. The vector of claim 1, wherein the AIMP2-DX2 gene has a nucleotide sequence at least 90% identical to a nucleotide sequence of SEQ ID NO:1.

8. The vector of claim 7, wherein the AIMP2-DX2 gene has a nucleotide sequence of SEQ ID NO:1.

9. The vector of claim 1, wherein the miR-142 target nucleic acid comprises a nucleotide sequence comprising ACACTA.

10. The vector of claim 9, wherein the miR-142 target nucleic acid comprises a nucleotide sequence comprising ACACTA and 1-17 additional contiguous nucleotides of SEQ ID NO:5.

11. The vector of claim 1, wherein the miR-142 target nucleic acid comprises a nucleotide sequence at least 50% identical to a nucleotide sequence of SEQ ID NO:5 (TCCATAAAGTAGGAAACACTACA).

12. The vector of claim 11, wherein the miR-142 target nucleic acid comprises a nucleotide sequence of SEQ ID NO:5.

13. The vector of claim 1, wherein the miR-142 target nucleic acid comprises a nucleotide sequence comprising ACTTTA.

14. The vector of claim 13, wherein the miR-142 target nucleic acid comprises a nucleotide sequence comprising ACTTTA and 1-15 additional contiguous nucleotides of SEQ ID NO:7.

15. The vector of claim 1, wherein the miR-142 target nucleic acid comprises a nucleotide sequence at least 50% identical to a nucleotide sequence of SEQ ID NO:7 (AGTAGTGCTTTCTACTTTATG).

16. The vector of claim 15, wherein the miR-142 target nucleic acid comprises a nucleotide sequence of SEQ ID NO:7.

17. The vector of claim 1, wherein the miR-142 target nucleic acid is repeated 2-10 times.

18. The vector of claim 1, wherein the vector is a viral vector.

19. The vector of claim 18, wherein the viral vector is an Adenovirus, Adeno-associated virus, Lentivirus, Retrovirus, Human immunodeficiency virus (HIV), MLU (Murine leukemia virus), ASLV (Avian sarcoma/leukosis), SNV (Spleen necrosis virus), RSV (Rous sarcoma virus), MMTV (Mouse mammary tumor virus), or Herpes simplex virus vector.

20. The vector of claim 18, wherein the viral vector is an adeno-associated virus (AAV), adenovirus, lentivirus, retrovirus, vaccinia virus, or herpes simplex virus vector.

21. A method of treating a neuronal disease in a subject in need thereof, comprising administering the vector of claim 1.

22. The method of claim 21, wherein the neuronal disease is amyotrophic lateral sclerosis (ALS), Alzheimer's disease, Parkinson's disease, retinal degeneration, mild cognitive impairment, multi-infarct dementia, fronto-temporal dementia, dementia with Lewy bodies, Huntington's disease, degenerative neural disease, metabolic cerebral disorders, depression, epilepsy, multiple sclerosis, cortico-basal degeneration, multiple system atrophy, progressive supranuclear palsy, dentatorubropallidoluysian atrophy, spinocerebella ataxia, primary lateral sclerosis, spinal muscular atrophy, or stroke.

23. The method of claim 22, wherein the neuronal disease is ALS.

24. The method claim 23, wherein the treatment improves motor activity or prolongs lifespan of the subject.

25. The method of claim 21, wherein the vector is administered to the brain or spinal cord.

26. The method of claim 25, wherein the vector is administered to the brain by stereotaxic injection.


SEQUENCE LISTING (9 sequences)

SEQ ID NO: 1 - DNA, 756 bp, Homo sapiens
atgccgatgtaccaggtaaagccctatcacgggggcggcgcgcctctccgtgtggagcttcccacctgcatgtaccggctccccaacgtgcacggcaggagctacggcccagcgccgggcgctggccacgtgcaggattacggggcgctgaaagacatcgtgatcaacgcaaacccggcctcccctcccctctccctgcttgtgctgcacaggctgctctgtgagcacttcagggtcctgtccacggtgcacacgcactcctcggtcaagagcgtgcctgaaaaccttctcaagtgctttggagaacagaataaaaaacagccccgccaagactatcagctgggattcactttaatttggaagaatgtgccgaagacgcagatgaaattcagcatccagacgatgtgccccatcgaaggcgaagggaacattgcacgtttcttgttctctctgtttggccagaagcataatgctgtcaacgcaacccttatagatagctgggtagatattgcgatttttcagttaaaagagggaagcagtaaagaaaaagccgctgttttccgctccatgaactctgctcttgggaagagcccttggctcgctgggaatgaactcaccgtagcagacgtggtgctgtggtctgtactccagcagatcggaggctgcagtgtgacagtgccagccaatgtgcagaggtggatgaggtcttgtgaaaacctggctccttttaacacggccctcaagctccttaagtga

SEQ ID NO: 2 - protein, 251 aa, Homo sapiens
MPMYQVKPYHGGGAPLRVELPTCMYRLPNVHGRSYGPAPGAGHVQDYGALKDIVIDADPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLTSVLQQIGGCSVTVPANVQRWMRSCENLAPFNTALKLLK

SEQ ID NO: 3 - DNA, 936 bp, Homo sapiens
atgccgatgtaccaggtaaagccctatcacgggggcggcgcgcctctccgtgtggagcttcccacctgcatgtaccggctccccaacgtgcacggcaggagctacggcccagcgccgggcgctggccacgtgcaggaagagtctaacctgtctctgcaagctcttgagtcccgccaagatgatattttaaaacgtctgtatgagttgaaagctgcagttgatggcctctccaagatgattcaaacaccagatgcagacttggatgtaaccaacataatccaagcggatgagcccacgactttaaccaccaatgcgctggacttgaattcagtgcttgggaaggattacggggcgctgaaagacatcgtgatcaacgcaaacccggcctcccctcccctctccctgcttgtgctgcacaggctgctctgtgagcacttcagggtcctgtccacggtgcacacgcactcctcggtcaagagcgtgcctgaaaaccttctcaagtgctttggagaacagaataaaaaacagccccgccaagactatcagctgggattcactttaatttggaagaatgtgccgaagacgcagatgaaattcagcatccagacgatgtgccccatcgaaggcgaagggaacattgcacgtttcttgttctctctgtttggccagaagcataatgctgtcaacgcaacccttatagatagctgggtagatattgcgatttttcagttaaaagagggaagcagtaaagaaaaagccgctgttttccgctccatgaactctgctcttgggaagagcccttggctcgctgggaatgaactcaccgtagcagacgtggtgctgtggtctgtactccagcagatcggaggctgcagtgtgacagtgccagccaatgtgcagaggtggatgaggtcttgtgaaaacctggctcctttt

SEQ ID NO: 4 - DNA, 207 bp, Homo sapiens
gaagagtctaacctgtctctgcaagctcttgagtcccgccaagatgatattttaaaacgtctgtatgagttgaaagctgcagttgatggcctctccaagatgattcaaacaccagatgcagacttggatgtaaccaacataatccaagcggatgagcccacgactttaaccaccaatgcgctggacttgaattcagtgcttgggaag

SEQ ID NO: 5 - DNA, 23 bp, Artificial Sequence
  Nucleotide sequence of miR-142-3p
tccataaagtaggaaacactaca

SEQ ID NO: 6 - DNA, 132 bp, Artificial Sequence
  Nucleotide sequence of 4 repeats of miR-142-3p
ccagaagcttgctagctccataaagtaggaaacactacatcactccataaagtaggaaacactacagatatctccataaagtaggaaacactacatcactccataaagtaggaaacactacaaagcttgtag

SEQ ID NO: 7 - DNA, 21 bp, Artificial Sequence
  Nucleotide sequence of miR-142-5p
agtagtgctttctactttatg

SEQ ID NO: 8 - DNA, 24 bp, Artificial Sequence
  AIMP2 variant Forward primer
ctggccacgtgcaggattacgggg

SEQ ID NO: 9 - DNA, 20 bp, Artificial Sequence
  AIMP2 variant reverse primer
aagtgaatcccagctgatag